(19) United States
(12) Patent Application Publication
(10) Pub. No.: US 2008/0260677 A1
(43) Pub. Date: Oct. 23, 2008

(54) Transdermal Topical Composition and Its Uses

(76) Inventor: Anant K. Pandya, Surrey (GB)

Correspondence Address: CATALYST LAW GROUP, APC, 9710 SCRANTON ROAD, SUITE S-170, SAN DIEGO, CA, 92121, US

(21) Appl. No.: 11/909,050

(22) PCT Filed: Mar. 22, 2006
(86) PCT No.: PCT/GB06/01058

§ 371 Date: Jul. 1, 2008

(30) Foreign Application Priority Data
Mar. 24, 2005 (GB) 0506141.1

(51) Int. Cl. A61K 31/765; A61K 31/196; A61K 31/573; A61Q 19/00

(52) U.S. Cl. 424/78.05; 514/567; 514/171

(57) ABSTRACT

An emollient component is provided in a composition, suitable for topical application to skin, comprising a fugitive solvent base comprising at least one alcohol. One advantage of the invention is that the irritancy potential of the composition due to the alcoholic fugitive solvent base is reduced.

The present invention relates to a composition for topical application to the skin. In particular, the invention relates to reducing the irritancy potential of topical compositions involving alcoholic fugitive solvents.

The skin is the largest organ of the human body. It has an important role in protecting the body from mechanical injury, water loss and the entry of harmful agents (e.g. disease-causing bacteria). It is also a sensory organ, containing receptors sensitive to pain, temperature and pressure. In warm-blooded animals, it helps regulate body temperature.

The skin is composed of two layers, the epidermis and the dermis. The epidermis has three layers, the outermost of which is called the stratum corneum which is a layer of dead keratinised cells forming a water-resistant barrier between the external environment and the living cells of the skin. The stratum corneum provides the first and most significant barrier to ingress of agents, for example pharmaceutically active agents, through the skin. In addition, the skin is constantly regenerating which makes prolonged application of such agents difficult.

Considerable effort has been invested over decades to overcome the stratum corneum barrier. Current topical preparations that target local effect are primarily available as semi-solid preparations consisting of creams, ointments, pastes, foams and gels. The mechanism of action is usually by passive diffusion of the active from a composition provided on the skin. Such compositions are usually greasy or powdery and frequently come into contact with clothing. Such contact reduces the effective dose applied and causes stains and/or greasiness on the skin and/or the clothes of the subject and on any other material with which the composition may come in contact. These factors affect patient morale and can result in patient non-compliance with use of a medication. Oily residues on the skin can also in some cases hinder drug absorption.

The dosage of existing topical compositions is usually provided empirically in terms of unit area of coverage. Such provision frequently results in under or over dosing.

Topical compositions are often constantly in contact with the epidermis of the skin which may result in irritation, particularly in people with skin that is more sensitive than normal.

U.S. Pat. No. 4,820,724 discloses a solvent carrier system for the topical application of pharmaceutically active compounds, e.g. antifungal agents. The solvent carrier system comprises a first solvent phase of a relatively high boiling solvent and a second solvent phase of a relatively low boiling solvent. When applied topically, the relatively low boiling solvent evaporates leaving a concentrated solution of the active in the relatively high boiling solvent. The increase in concentration of the active compound assists penetration of the active compound into the skin. U.S. Pat. No. 4,850,724 exemplifies the use of a composition comprising 1 wt % griseofulvin, 10 wt % benzyl alcohol, 40 wt % acetone and 50 wt % (sic) isopropyl alcohol in the treatment of tinea pedis infection.

One disadvantage of the solvent carrier system disclosed in U.S. Pat. No. 4,820,724 is that the organic solvents (and in particular, isopropyl alcohol), can cause irritation, particularly if the patient has sensitive skin or suffers from conditions in which the skin is raw, split or has lesions. Examples of such conditions include eczema, psoriasis, abrasions and infections of the skin.

The high irritancy potential of such alcoholic compositions not only increases the risk of patient non-compliance but also reduces the range of possible uses of the compositions. In such cases, a composition having a non-alcoholic solvent vehicle would be used. However, these compositions tend to be greasy or leave a residue and suffer from the disadvantages discussed above.

It is desirable to be able to topically administer pharmaceutically active agents directly and efficiently to the skin of affected areas, leaving as little residue as possible. In this connection and in view of the prior art, there is still a need for a topical composition that overcomes the above-mentioned difficulties and disadvantages. It is particularly desirable to develop a composition for topical application to the skin that retains the benefits of using alcohols as fugitive solvents but with reduced irritancy potential.

According to a first aspect of the present invention, there is provided use of an emollient component to reduce irritancy potential of a fugitive solvent comprising at least one alcohol in a therapeutic composition for application to skin.

According to a second aspect of the present invention, there is provided a composition for topical application to skin comprising:
a fugitive solvent base comprising at least one alcohol; and
an emollient component.

One advantage of the present invention is its universal application. The presence of the emollient component allows the use of an alcoholic fugitive solvent base in the composition thereby enabling the achievement of all of the advantages arising from the use of fugitive solvents but with reduced irritancy potential. For example, preferred compositions of the present invention have particular application in cases where the patient has sensitive skin or suffers from conditions in which the skin is raw, split or has lesions. Examples of conditions that may be treated using preferred compositions of the present invention include eczema, psoriasis, abrasions and infections of the skin. Thus, the positive combination of beneficial features of compositions of the present invention increases the range of potential uses to which compositions comprising alcoholic fugitive solvents may be applied.

The emollient component may be a single compound or a mixture of compounds. Suitable compounds for use in the emollient component include glycols (e.g. propylene glycol); polyglycols; fatty acids and their derivatives such as fatty acid esters; vegetable oils; and silicones. Preferably, the emollient component comprises at least one silicone although mixtures of silicones may also be used.

Examples of suitable silicones include polydimethylsiloxanes (e.g. dimethicones; and cyclomethicones) and oligodimethylsiloxanes (e.g. hexa-methyldisiloxane (“HMDS”) and octamethyltrisiloxane (“OMTS”)). Simethicones (i.e. dimethicones activated with silicon dioxide) may also be used.

Dimethicones are graded according to their viscosities. Suitable dimethicones may have a viscosity from about 20 centiStokes (“cSt”) to about 1250 cSt, preferably from about 20 cSt to about 1000 cSt. Preferred dimethicones have a viscosity of about 20 cSt, about 100 cSt or about 350 cSt. The most preferred dimethicone is either Dimethicone USP NF or Dimethicone Ph.Eur. The grading for cyclomethicones is less well defined. The preferred cyclomethicone is Cyclomethicone USP NF or Cyclomethicone Ph.Eur.

The emollient component is typically present in an amount of from about 5 wt % to about 50 wt %, preferably from about 10 wt % to about 40 wt % and more preferably from about 25 wt % to about 35 wt %, calculated on the basis of the total weight of the composition. In preferred embodiments, the emollient component is present in an amount of about 20 wt % or about 30 wt % of the total composition.

The compositions of the present invention are suitable for use as vehicles for the topical application of specific compounds to the skin using pharmaceutical, nutraceutical, cosmetic or veterinary preparations. Such topical application enables the specific compounds to have a local effect on or in the region of particular areas of the skin.

The composition will usually further comprise at least one active compound and, optionally, at least one penetration enhancer.

The or at least one active compound may be a pharmacologically active compound. A “pharmacologically active compound” is a compound that has a therapeutic effect on the human or animal body in the treatment or prevention of a condition.

Suitable pharmacologically active compounds may be selected from:
non-steroidal anti-inflammatory drug (“NSAID”) compounds such as diclofenac; ibuprofen; piroxicam; ketoprofen; naproxen; salicylate compounds; and COX-1 and COX-2 inhibitors, e.g. celecoxib;
glucocorticosteroids such as cortisone; hydrocortisone; betamethasone; beclomethasone; budesonide; triamcinolone and prednisolone;
immunosuppressants such as cyclosporin; methotrexate; pimecrolimus; and tacrolimus;
antibiotic agents such as fusidic acid; mupirocin; polymixins; tetracycline and its derivatives; cephalosporins; cephamycins; beta-lactam; clindamycin; aminoglycosides; vancomycin; teicoplanin; linezoid; streptomycins; sulphanoamides; metronidazole and its derivatives; benzoyl peroxide; and quinolones;
antifungal agents such as amphoteracin; nystatin; imidazoles; triazoles; grisofulvin; allylamines; azoles; and amorolfine;
antiseptic agents such as chlorhexidine; cetrimide; and povidone;
antiviral agents such as nucleoside analogues, e.g. acyclovir and famcyclovir;
local anaesthetics such as lidocaine;
short-acting antihistamines such as mepyramine and diphenhydramine; and long-acting anti-histamines such as astemizole and azelastine;
agents for treating pruritus such as doxepin;
agents for treating actinic keratosis and similar pre-cancerous and cancerous conditions of the skin such as diclofenac; tretinoin and other retinoids;
skin cleansers and desloughing agents such as hydrogen peroxide and benzoic acid;
agents for wound management such as alginates and hydrogels; agents for treating circulatory disorders such as heparin and heparinoid;
agents for treating hyperhidrosis such as aluminium salts and glycopyronium;
anti-acne agents such benzyl peroxide and antibiotics such as erythromycin and clindamycin;
Anti-rheumatic agents such as topical NSAIDs, e.g. diclofenac; piroxicam; Ibuprofen; and ketoprofen;
rubefacients such as camphor; ethyl nicotinate; and methyl salicylate;
agents for treating warts and calluses such as salicylic acid, lactic acid, gluteraldehyde, podophyllum;
other agents such as vitamin D and its analogues; vitamin A and its analogues; retinoids; dithranols; coal tar; nicotine and its derivatives; and
colchicine for the treatment of gout and psoriasis.

The present invention has particular application for the topical administration of NSAIDs (in particular, diclofenac, ibuprofen and piroxicam); steroids (in particular, hydrocortisone); antibiotics (in particular, fusidic acid); doxepin; and colchicine.

The or at least one active compound may be a nutraceutically active compound. A “nutraceutically active compound” is a compound, derived from a natural origin (animal or vegetable) that has a beneficial and/or therapeutic effect on the human or animal body in the treatment of a condition. Such compounds may be regarded as nutrients.

Suitable nutraceutically active compounds may be natural products extracted from animals or vegetables. Examples of suitable nutraceutically active compounds include:
carotenoids such as lycopene, lutein, astaxanthin and β-carotene;
glucosamine or N-acylglucosamine;
ubiquinone;
Vitamins such as vitamins A, C, D and E;
Rosmarinic acid;
Honokiol;
Magnolol;
Chlorogenic acid;
Oleuropein;
Methylsulphonylmethane (“MSM”);
Chondroitin;
Boswellin and boswellic acid;
Escin and esculin;
Tumeric extracts such as curcuminoids and tetrahydrocurcuminoids;
Gingerol and gingerone;
Triterpenes such as ursolic acid and oleanolic acid;
Diterpenes such as asiaticoside, sericoside and ruscogenins;
Hydroxycitric acid (“HCA”) and niacinamide hydroxycitrate;
Trigonellin; and
Corosolic acid.

Pharmacologically acceptable derivatives (including salts) of the pharmacologically or nutraceutically active compounds may also be used.

The composition may comprise one or more components having a cosmetic effect. Such components include collagen and retinols.

The pharmacologically active compounds, the nutraceutically active compounds and the cosmetic components may either be used alone or in any combination.

The active compound is present in preferred embodiments in a therapeutic amount, e.g. an amount calculated to enable a beneficial and/or therapeutic effect on the human or animal body with the correct dosage. The active compound(s) is typically present in an amount of from about 0.1 wt % to about 10 wt % based on the total weight of the composition. In some preferred embodiments, the amount is from about 0.5 wt % to 5 wt % and more preferably from about 1 wt % to about 3 wt %, for example about 1 wt % or about 2 wt %.

The compositions may further comprise at least one penetration enhancer. Examples of suitable penetration enhancers for use in preferred compositions of the present invention include benzyl alcohol; silicone based enhancers such as HMDS and OMTS; azone; and triglyceride fatty acids. Non-silicone penetration enhancers are preferred with benzyl alcohol being particularly preferred.

Where present, the penetration enhancer is typically present in an amount of from about 1 wt % to about 15 wt % and preferably from about 5 wt % to about 15 wt %, based on the total weight of the composition. In preferred embodiments, the penetration enhancer is present in an amount of about 5 wt % or about 10 wt %.

The purpose of the fugitive solvent base is to provide a medium by which the active(s) is administered to the skin and then to evaporate leaving the active(s) concentrated in the residue on the surface of the skin.

The fugative solvent base comprises an alcohol. Preferably, the fugitive solvent base comprises two components selected from the group consisting of C₁-C₄ alcohols and C₁-C₄ ketones. Suitable alcohols are, preferably, monohydric aliphatic alcohols such as methyl alcohol; ethyl alcohol; propyl alcohol; isopropyl alcohol; butyl alcohol; and isobutyl alcohol. Isopropyl alcohol is preferred. Mixtures of alcohols may also be suitable. For example, the fugitive solvent may consist of a mixture of isopropyl alcohol and ethyl alcohol.

Ketones such as acetone; propanone; or butanone may also be present in the fugitive solvent base. In these embodiments, acetone is preferred. In some embodiments, the fugitive solvent base may consist of a mixture of monohydric aliphatic alcohol and a ketone. For example, the fugitive solvent base may consist of a mixture of isopropyl alcohol and acetone.

The choice of components for the fugitive solvent base depends on the stability of the active(s) in the composition. Salts of some active(s) react with ketones. For example, some nicotine metabolites react with acetone. Thus, ketones are not suitable components for the solvent base where the active is such a compound. In such cases, a mixture of monohydric aliphatic alcohols might be used.

In embodiments of the present invention in which the fugitive solvent base is a mixture of monohydric aliphatic alcohol and ketone, the monohydric aliphatic alcohol is typically present in an amount of from about 20 wt % to about 50 wt % and preferably from about 25 wt % to about 40 wt %, based on the total weight of the composition. The ketone is typically present in an amount of from about 20 wt % to about 50 wt % and preferably from about 25 wt % to about 35 wt %, based on the total weight of the composition.

The compositions of the present invention may be in any form suitable for topical application to the skin. Suitable forms include sprayable liquids; gels; liquids that may be applied using a roll-on device; lacquers; and sustained release matrices of transdermal delivery devices such as patches.

The compositions of the present invention have particular application in the topical administration of active compounds for a local effect.

According to a third aspect of the present invention, there is provided a dispenser comprising a container containing a dispensable composition according to the second aspect and dispensing means for dispensing the composition. Preferably, the dispensing means dispenses a metered dose of the composition. One advantage of these embodiments is that the risk of over or under dosing of the active(s) is reduced.

In one preferred embodiment, the composition is in the form of a sprayable liquid that may be administered using a spray dispenser. A suitable spray dispenser comprises a container containing a sprayable composition according to the first aspect and dispensing means suitable for dispensing the composition in the form of a spray.

In another preferred embodiment, the composition is in the form of a liquid that may be administered using a roll-on device. A suitable roll-on device comprises a container containing a liquid composition according to the first aspect and roller dispensing means suitable for dispensing the composition.

In other preferred embodiments, the composition is applied in the form of a lacquer.

According to a fourth aspect of the present invention, there is provided a therapeutic composition for topical application to skin comprising:

at least one active compound selected from the group consisting of pharmacologically and nutraceutically active compounds;

a fugitive solvent base comprising at least one alcohol; and

an emollient component,

for use in the treatment of the human or animal body by therapy. The therapeutic composition may have any of the features described above in any appropriate combination.

According to a fifth aspect of the present invention, there is provided a method of reducing irritancy potential of a fugitive solvent comprising at least one alcohol in a therapeutic composition for application to the skin, said method comprising incorporating an emollient component in the composition.

Therapeutic compositions of the present invention may be used to treat or prevent a wide variety of conditions depending on the choice of active compound or combination of active compounds. Methods of treatment or prophylaxis of the conditions comprise administering topically to an area of skin a therapeutic amount of an appropriate composition according to the present invention. In this connection,
Eczema or dermatitis may be treated with steroids or NSAIDs. An example of a suitable steroid is hydrocortisone and an example of a suitable NSAID is diclofenac;
Psoriasis may be treated with steroids, vitamins or colchicine. An example of a suitable steroid is hydrocortisone and an example of a suitable vitamin is vitamin A or vitamin B;
Actinic keratosis, pre-cancerous or cancerous lesions, melanomas and mycosis fugoides may be treated using immunosuppressants or NSAIDs. An example of a suitable immunosuppressant is cyclosporin and an example of a suitable NSAID is diclofenac;
Infections may be treated using anti-infective agents, e.g. anti-biotics such as fusidic acid; anti-viral agents such as acyclovir; and anti-fungal agents such as terbinafine. Infections may be prevented using anti-septic agents such as chlorhexidine;
Pruritis may be treated using doxepin;
Wounds may be treated using alginates or hydrogels;
Circulatory disorders may be treated using heparinoid;
Hyperhidrosis may be treated using aluminium salts or glycopyronium;
Acne may be treated using benzyl peroxide or anti-biotics such as erythromycin or clindamycin;

Rheumatism may be treated using NSAIDs such as diclofenac;
Warts and calluses may be treated using salicylic acid, lactic acid, glutaldehyde or podophyllum;
Gout may be treated using colchicine;
Arthritis may be treated using anti-inflammatory agents such as ibuprofen;
Keloids may be treated using interferon; verapamil; bleomycin; 5-fluorouracil (“5-FU”); retinoic acid; imiquimod; tacrolimus; and botulinum toxin; and
Vitiligo may be treated using psoralen; topical 4-methoxyphenol; fluticasone propionate; methylprednisolone; and calcipotriol.

The invention will now be described with reference to the following example.

### EXAMPLE

A study was performed to compare the irritancy potential of two series of formulations according to the present invention against commercially available formulations. The first series comprised a steroid (hydrocortisone) and the second series comprised a NSAID (diclofenac).

In each test, a synthetic skin (Reconstituted Human Epidermal (RHE) model from SkinEthic Laboratories, Nice, France) was exposed for 15 minutes to the test formulation and then subjected to a 42 hour post treatment incubation period. The synthetic skin consists of an airlifted, living, multi-layered epidermal tissue construct, produced in polycarbonate inserts in a serum-free and chemically defined medium, featuring normal ultra-structure that is functionally equivalent to human epidermis in vivo. The test formulations were applied directly to the culture surface, at air interface, so that undiluted and/or end use dilutions could be tested directly.

Toxicity was determined using a Multiple Endpoint Analysis (MEA) approach for cell viability (MTT reduction test), histopathology, and inflammatory mediator release.

### Hydrocortisone Formulations

Five hydrocortisone formulations were prepared having the compositions indicated as T1 to T5 in Table 1. A commercially available hydrocortisone ointment (EFCORTELAN; GlaxoSmithKline, Stockley Park West, Uxbridge Middlesex, UB11 1BT, UK) was used as a comparative composition (T6). The composition of T6 is 1 wt % hydrocortisone in white soft paraffin BP and liquid paraffin. The results are indicated in Table 1.

TABLE 1

| HYDROCORTISONE | | | | | | |
|---|---|---|---|---|---|---|
| FORMULATION | T1 | T2 | T3 | T4 | T5 | T6 |
| Hydrocortisone | 0 | 0 | 0 | 0 | 1 | |
| Benzyl alcohol | 5 | 5 | 5 | 5 | 5 | |
| Dimethicone | | 30 | 20 | 20 | 20 | |
| Heaxamethyldisiloxane | | | | 10 | 10 | |
| Cyclomethicone USP | | | 10 | | 10 | |
| Isopropyl alcohol | 48 | 33 | 33 | 33 | 27 | |
| Acetone | 47 | 32 | 32 | 32 | 27 | |
| Irritancy ranking | | | | | | |
| 1 = least irritant | | | | | | |
| 6 = most irritant | | | | | | |
| Cell Viability (MTT) | 5 | 1 | 2 | 3 | 4 | 6 |
| Histology - Effect | L | L | L | L | M | M |
| L = Little | | | | | | |
| M = Slight to moderate | | | | | | |
| S = Severe | | | | | | |
| OVERALL RANKING | 4 | 1 | 2 | 3 | 5 | 6 |
| 1 = least irritant and 6 most irritant | | | | | | |

The results indicate an overall viability ranking of:
T2>T3>T4>T1>T5>T6
   with T2 (30 wt % dimethicone as the emollient component) being the most viable and T6 (the commercially available ointment formulation) being the least viable in terms of reducing the irritancy potential of the alcoholic fugitive solvent base.

### Diclofenac Formulations

Four diclofenac formulations were prepared having the compositions indicated as T7 to T10 in Table 2. A commercially available diclofenac gel (VOLTAROL EMULGEL; Novartis Pharmaceuticals UK Ltd., trading as Geigy Pharmaceuticals, Frimley Business Park, Frimley, Surrey, GU16 7SR) was used as a comparative composition (T11). The composition of T11 is 1.16 wt % diclofenac sodium (=1 g diclofenac), diethylamine, carbomer, macrogol cetostearyl ether, cocyl caprylocaprate, isopropyl alcohol, liquid paraffin heavy, perfume cream 45, polypropylene glycol dist., and water. The results are indicated in Table 2.

TABLE 2

| | | | | | T11 |
|---|---|---|---|---|---|
| DICLOFENAC | | | | | Marketed |
| FORMULATION | T7 | T8 | T9 | T10 | Gel |
| Diclofenac | 2 | 2 | 2 | 2 | 1 |
| Benzyl alcohol | 10 | 10 | 10 | 10 | |
| Dimethicone | | | 20 | 10 | |
| Heaxamethyldisiloxane | | 10 | 20 | | |
| Cyclomethicone USP | | | | | |
| Isopropyl alcohol | 48 | 42 | 26 | 42 | |
| Acetone | 40 | 36 | 22 | 36 | |
| Irritancy ranking | | | | | |
| 1 = least irritant and | | | | | |
| 6 = most irritant | | | | | |
| Cell Viability (MTT) | 2 | 3 | 5 | 1 | 4 |
| Histology - Effect | L | M | M | L | M |
| L = Little | | | | | |
| M = Slight to moderate | | | | | |
| S = Severe | | | | | |
| OVERALL RANKING | 2 | 3 | 5 | 1 | 4 |
| 1 = least irritant and 6 most irritant | | | | | |

The results indicate an overall viability ranking of:
T10>T7>T8>T11>T9
   with T10 (10 wt % dimethicone as the emollient component) being the most viable and T9 being the least viable in terms of reducing the irritancy potential of the alcoholic fugitive solvent base.

Throughout the specification, the term “means” in the context of means for carrying out a function, is intended to refer to at least one device adapted and/or constructed to carry out that function.

It will be appreciated that the invention is not restricted to the details described above with reference to the preferred embodiments but that numerous modifications and variations can be made without departing from the spirit or scope of the invention as defined by the following claims.

1. A method of using an emollient component to reduce irritancy potential of a fugitive solvent comprising at least one alcohol in a composition for application to skin, the method comprising applying the composition to skin.

2. The method as claimed in claim 1 wherein the emollient component comprises at least one of a glycol; a polyglycol; a fatty acid; a fatty acid ester; a vegetable oil; or a silicone.

3. The method as claimed in claim 1 wherein the emollient component comprises at least one silicone.

4. The method as claimed in claim 1 wherein the emollient component is selected from polydimethylsiloxanes; oligodimethylsiloxanes; or a mixture thereof.

5. The method as claimed in claim 1 wherein the emollient component is dimethicone.

6. The method as claimed in claim 1 wherein the fugitive solvent base comprises two components selected from the group consisting of C₁-C₄ alcohols and C₁-C₄ ketones.

7. The method as claimed in claim 1 wherein the composition is a therapeutic composition.

8. The method as claimed in claim 1 wherein the composition further comprises at least one active compound.

9. The method as claimed in claim 8 wherein the or at least one active compound is a pharmacologically active compound.

10. The method as claimed in claim 9 wherein the pharmacologically active compound is a NSAID.

11. The method as claimed in claim 10 wherein the NSAID is diclofenac.

12. The method as claimed in claim 8 wherein the pharmacologically active compound is a steroid.

13. The method as claimed in claim 12 wherein the steroid is hydrocortisone.

14. The method as claimed in claim 8 wherein the or at least one active compound is a nutraceutically active compound.

15. The method as claimed in claim 8 wherein the active compound has a local effect.

16. The method as claimed in claim 1 wherein the composition further comprises at least one penetration enhancer.

17. A composition for topical application to skin comprising:
a fugitive solvent base comprising at least one alcohol; and
an emollient component.

18. (canceled)

19. The composition as claimed in claim 17 wherein the fugitive solvent base comprises two components selected from the group consisting of C₁-C₄ alcohols and C₁-C₄ ketones.

20. A therapeutic composition for topical application to skin comprising:
at least one active compound;
a fugitive solvent base comprising at least one alcohol; and
an emollient component,
for use in the treatment of the human or animal body by therapy.

21. A dispenser comprising a container containing a dispensable composition as defined in claim 17 and dispensing means for dispensing the composition.

22.-23. (canceled)

24. A dispenser comprising a container containing a dispensable composition as defined in claim 20 and dispensing means for dispensing the composition.
